(19) United States
(12) Patent Application Publication
(10) Pub. No.: US 2025/0331811 A1
(43) Pub. Date: Oct. 30, 2025

(54) ULTRASONIC PROBE AND ULTRASONIC DEVICE

(71) Applicant: SHENZHEN MINDRAY BIO-MEDICAL ELECTRONICS CO., LTD., Shenzhen (CN)

(72) Inventors: Jingwen LI, Shenzhen (CN); Ming TANG, Shenzhen (CN)

(73) Assignee: SHENZHEN MINDRAY BIO-MEDICAL ELECTRONICS CO., LTD., Shenzhen (CN)

(21) Appl. No.: 19/193,952

(22) Filed: Apr. 29, 2025

(30) Foreign Application Priority Data
Apr. 30, 2024 (CN) 202410543721.0

(51) Int. Cl. A61B 8/12; A61B 8/00

(52) U.S. Cl. CPC A61B 8/12; A61B 8/4455; A61B 8/4461; A61B 8/483; A61B 2560/0462; A61B 2562/168

(57) ABSTRACT

Disclosed are an ultrasonic probe and an ultrasonic device. At least a portion of the surface on the swing trajectory of the transducer that faces the coupling fluid compensating member is a first profiled surface, the coupling fluid compensating member has at least a first profiling surface facing the first profiled surface. The shape of the first profiling surface is designed based on the shape of the first profiled surface to ensure that the two surfaces are of the same shape. The coupling fluid compensating member can thus be as close as possible to the swing trajectory of the transducer without affecting the swing of the transducer. This can reduce the size of the coupling fluid cavity, and consequently the volume of the coupling fluid, thereby lightening the weight of the ultrasonic probe.

### TECHNICAL FIELD

The present disclosure relates to the field of medical devices, in particular to the structure of an ultrasonic probe.

### BACKGROUND

Ultrasonic devices are extremely important medical instruments in the medical field, enabling doctors to obtain images of tissues within the body of an object under examination, aiding in diagnostic and therapeutic procedures. To achieve superior visualization, some ultrasonic devices are equipped with 3D and 4D ultrasonic probes. These probes are capable of generating 3D images (three-dimensional images). In order to generate a 3D image, the transducer in a 3D or 4D ultrasonic probe is designed to swing around an axis, allowing it to capture two-dimensional ultrasound images (B-mode images) at various swing angles. These 2D images are subsequently stitched together to generate a comprehensive 3D image.

In a 3D or 4D ultrasonic probe, to ensure smooth transmission of ultrasonic waves, the gap between the transducer and the acoustic window needs to be filled with coupling fluid (i.e., medical ultrasonic couplant). When the coupling fluid fills the entire coupling fluid cavity, the overall weight of the ultrasonic probe becomes relatively heavy, which is detrimental to the lightweight design of the probe, causing inconvenience and fatigue for operators. However, the transducer needs to swing freely within the coupling fluid cavity, preventing arbitrary reduction or alteration of the cavity size. Therefore, the challenge lies in reducing the weight of existing ultrasonic probes without impacting the swinging motion of the transducer.

### SUMMARY

The present disclosure provides ultrasonic probes and ultrasonic devices that are capable of reducing the weight of the ultrasonic probes without affecting the movement of the transducers through their structural improvements.

An ultrasonic probe provided in some embodiments of the present disclosure may include:
a transducer, configured to transmit and receive ultrasonic signals;
an acoustic window;
a transducer base, wherein the transducer is mounted on the transducer base;
a coupling fluid compensating member, wherein the acoustic window, the transducer base and the coupling fluid compensating member enclose a coupling fluid cavity filled with a coupling fluid, and the transducer is disposed in a swingable manner within the coupling fluid cavity; and
a transducer driving component, configured to drive the transducer to swing;
wherein a swing trajectory is generated during the swinging of the transducer, at least a portion of the coupling fluid compensating member is disposed between the swing trajectory and the transducer base, at least a portion of the surface of the swing trajectory that faces the coupling fluid compensating member serves as a first profiled surface, and the coupling fluid compensating member has at least one first profiling surface that faces the first profiled surface, where the first profiling surface and the first profiled surface are of the same shape and disposed opposite to each other.

According to the ultrasonic probe provided in these embodiments, since at least a portion of the surface of the swing trajectory of the transducer that faces the coupling fluid compensating member is configured as a first profiled surface, and the coupling fluid compensating member has at least a first profiling surface that faces the first profiled surface, as well as the shape of the first profiling surface being designed based on the shape of the first profiled surface to ensure that the two surfaces are of the same shape, the coupling fluid compensating member can thus be as close as possible to the swing trajectory of the transducer without affecting the swing of the transducer. This can reduce the size of the coupling fluid cavity, and consequently the volume of the coupling fluid, thereby lightening the weight of the ultrasonic probe.

In some embodiments, the shortest distance between any point on the first profiling surface and the corresponding point on the first profiled surface is equal.

In some embodiments, the shortest distance between any point on the first profiling surface and the corresponding point on the first profiled surface is less than or equal to 10 mm.

In some embodiments, the shortest distance between any point on the first profiling surface and the corresponding point on the first profiled surface is less than or equal to 2 mm.

In some embodiments, the surface of the transducer facing the transducer base is defined as a transducer bottom surface, at least a portion of the transducer bottom surface is concave towards the interior of the transducer to form an inner concave surface, and at least a portion of the coupling fluid compensating member extends into a region enclosed by the inner concave surface.

In some embodiments, each of the at least one first profiling surface is an arc surface having a swing axis of the transducer as its central axis.

In some embodiments, the coupling fluid compensating member is made of an elastic, soft material, allowing it to adaptively deform based on a pressure exerted by the coupling fluid onto the coupling fluid compensating member.

In some embodiments, the transducer base and the handle housing enclose an installation cavity for the transducer driving component, the transducer driving component is disposed within the installation cavity for the transducer driving component; the transducer base is provided with a vent communicating with the installation cavity for the transducer driving component, the coupling fluid compensating member has a chamber structure, the chamber structure is sealably covered over the vent of the transducer base and is in communication with the vent, thereby communicating an inner cavity of the chamber structure with the installation cavity for the transducer driving component.

In some embodiments, the transducer base has a protruding part that protrudes towards the transducer, the protruding part has a second profiling surface, and at least a portion of the surface of the swing trajectory that faces the protruding part serves as a second profiled surface, where the second profiling surface and the second profiled surface are of the same shape and disposed opposite to each other.

In some embodiments, the transducer driving component is disposed on the side of the transducer base away from the transducer, the side of the protruding part that faces the transducer driving component forms a accommodation cavity, and at least a portion of the transducer driving component is accommodated within the accommodation cavity.

In some embodiments, at least a portion of the surface of the transducer that faces the transducer base is concave inward to form the inner concave surface within the transducer itself, and the protruding part extends into a region enclosed by the inner concave surface.

An ultrasonic probe provided in some other embodiments of the present disclosure may include:
a transducer, configured to transmit and receive ultrasonic signals;
an acoustic window;
a transducer base, wherein the transducer is mounted on the transducer base, the acoustic window and the transducer base enclose a coupling fluid cavity filled with a coupling fluid, and the transducer is disposed in a swingable manner within the coupling fluid cavity; and
a transducer driving component, configured to drive the transducer to swing;
wherein the transducer base has a protruding part that protrudes towards the transducer, a swing trajectory is generated during the swing of the transducer, at least a portion of the surface of the swing trajectory that faces the protruding part serves as a second profiled surface, and the protruding part has a second profiling surface, where the second profiling surface and the second profiled surface are of the same shape and disposed opposite to each other.

According to the ultrasonic probe provided in these embodiments, since the transducer base has a protruding part that protrudes towards the transducer that can swing to generate the swing trajectory, where at least a portion of the surface of the swing trajectory that faces the protruding part serves as a second profiled surface, and the protruding part has a second profiling surface, as well as the shape of the second profiling surface being designed based on the shape of the second profiled surface to ensure that the two surfaces are of the same shape, the protruding part can thus be as close as possible to the swing trajectory of the transducer without affecting the swing of the transducer. This can reduce the size of the coupling fluid cavity, and consequently the volume of the coupling fluid, thereby lightening the weight of the ultrasonic probe.

In some embodiments, the shortest distance between any point on the second profiling surface and the corresponding point on the second profiled surface is equal.

In some embodiments, the shortest distance between any point on the second profiling surface and the corresponding point on the second profiled surface is less than or equal to 2 mm.

In some embodiments, the transducer driving component is disposed on a side of the transducer base away from the transducer, at least a portion of the surface of the transducer that faces the transducer base is concave inward to form an inner concave surface within the transducer itself, the protruding part extends into a region enclosed by the inner concave surface; the side of the protruding part that faces the transducer driving component forms a accommodation cavity, and at least a portion of the transducer driving component is accommodated within the accommodation cavity.

An ultrasonic probe provided in some other embodiments of the present disclosure may include:
a probe base;
a connecting housing, connected to the probe base;
an acoustic window, connected to the connecting housing;
a coupling fluid compensating member, wherein the probe base, the acoustic window,
the connecting housing and the coupling fluid compensating member enclosed a coupling fluid cavity filled with a coupling fluid;
a transducer base, disposed within the coupling fluid cavity;
a transducer, configured to transmit and receive ultrasonic signals, wherein the transducer is mounted on the transducer base and is capable of swinging within the coupling fluid cavity; and
a transducer driving component, configured to drive the transducer to swing;
wherein at least a portion of the inner side wall of the connecting housing serves as a third profiled surface, the coupling fluid compensating member has at least a third profiling surface that faces the third profiled surface, where the third profiling surface and the third profiled surface are of the same shape and disposed opposite to each other.

According to the ultrasonic probe provided in these embodiments, since at least a portion of the inner side wall of its connecting housing serves as a third profiled surface, and the coupling fluid compensating member has at least a third profiling surface that faces the third profiled surface, as well as the shape of the third profiling surface being designed based on the shape of the third profiled surface to ensure the two surfaces are of the same surface, the coupling fluid compensating member can thus be as close as possible to the inner side wall of the connecting housing without affecting the swing of the transducer. This can reduce the size of the coupling fluid cavity, and consequently the volume of the coupling fluid, thereby lightening the weight of the ultrasonic probe.

In some embodiments, the shortest distance between any point on the third profiling surface and the corresponding point on the third profiled surface is equal.

In some embodiments, the shortest distance between any point on the third profiling surface and the corresponding point on the third profiled surface is less than or equal to 10 mm.

In some embodiments, the shortest distance between any point on the third profiling surface and the corresponding point on the third profiled surface is less than or equal to 2 mm.

In some embodiments, the coupling fluid compensating member is made of an elastic, soft material, allowing it to adaptively deform based on a pressure exerted by the coupling fluid onto the coupling fluid compensating member.

In some embodiments, the connecting housing is elongated, and the coupling fluid compensating member is mounted on the probe base and extends into the connecting housing along the length direction of the connecting housing.

In some embodiments, the ultrasonic probe is an endocavity probe.

An ultrasonic probe provided in some other embodiments of the present disclosure may include:
a coupling fluid cavity structure, wherein the coupling fluid cavity structure encloses a coupling fluid cavity filled with a coupling fluid, and a portion of the coupling fluid cavity structure is a profiling structure that protrudes towards an interior of the coupling fluid cavity;
a transducer, disposed in a swingable manner within the coupling fluid cavity, wherein a swing trajectory is generated during the swing of the transducer; and
a transducer driving component, configured to drive the transducer to swing;
wherein at least a portion of a surface of the swing trajectory that faces the profiling structure serves as a profiled surface, and/or at least a portion of surfaces, other than the profiling structure, of walls of the coupling fluid cavity, is configured as a profiled surface; and the profiling structure has at least a profiling surface that faces the profiled surface, where the profiling surface and the profiled surface are of a same shape and disposed opposite to each other.

According to the ultrasonic probe provided in these embodiments, since a portion of its coupling fluid cavity structure is a profiling structure that protrudes towards the interior of the coupling fluid cavity, and at least a portion of the surface of the swing trajectory of the transducer that faces the profiling structure serves as a profiled surface, and/or at least a portion of the surfaces, excluding the profiling structure, of the cavity walls of the coupling fluid cavity, serves as a profiled surface, as well as the shape of the profiling surface being designed based on the shape of the profiled surface to ensure that the two surfaces are of the same shape, the profiling structure can thus be as close as possible to the swing trajectory of the transducer or to the surfaces, excluding the profiling structure, of the cavity wall of the coupling fluid cavity, without affecting the swing of the transducer. This can reduce the size of the coupling fluid cavity, and consequently the volume of the coupling fluid, thereby lightening the weight of the ultrasonic probe.

In some embodiments, the profiling structure is a coupling fluid compensating member that is capable of adaptively deforming based on a pressure exerted by the coupling fluid onto the coupling fluid compensating member.

In some embodiments, the shortest distance between any point on the profiling surface and the corresponding point on the profiled surface is equal.

In some embodiments, the shortest distance between any point on the profiling surface and the corresponding point on the profiled surface is less than or equal to 2 mm.

An ultrasonic device is also provided in some embodiments of the present disclosure. The ultrasonic device may include an ultrasonic host and an ultrasonic probe mentioned in the above any embodiment, wherein the ultrasonic host is provided with a control unit configured to control operations of the ultrasonic probe.

According to the ultrasonic device provided in these embodiments, since it adopts the ultrasonic probe mentioned in the above any embodiment, the weight of the ultrasonic probe can be reduced without affecting the swing of the transducer.

### DESCRIPTION OF THE DRAWINGS

FIG. 1 schematically illustrates a partial external view of the ultrasonic probe in some embodiments of the present disclosure;

FIG. 2 schematically illustrates a cross-sectional view of the ultrasonic probe along its length direction in some embodiments of the present disclosure, wherein the ultrasonic probe may be an endocavity probe;

FIG. 3 schematically illustrates the internal structure of the ultrasonic probe in some embodiments of the present disclosure, with the acoustic window and the handle housing thereof presented in a cross-sectional view;

FIG. 4 schematically illustrates the internal structure of the ultrasonic probe in some embodiments of the present disclosure, with the acoustic window thereof presented in a cross-sectional view but without a protruding part on the transducer base of the ultrasonic probe;

FIG. 5 schematically illustrates the swing trajectory of the transducer and the profiling surface of the profiling structure in some embodiments of the present disclosure;

FIG. 6 schematically illustrates an external view of the coupling fluid compensating member in some embodiments of the present disclosure;

FIG. 7 schematically illustrates a structural view of the transducer base, the coupling fluid compensating member and the transducer driving component in some embodiments of the present disclosure;

FIG. 8 schematically illustrates a cross-sectional view of the protruding part of the transducer base and the coupling fluid compensating member in some embodiments of the present disclosure; and

FIG. 9 schematically illustrates an exploded view of the transducer base, the coupling fluid compensating member and the transducer driving component in some embodiments of the present disclosure.

### DETAILED DESCRIPTION

The present disclosure will be further described in detail below through specific embodiments with reference to the accompanying drawings. Common or similar elements are referenced with like or identical reference numerals in different embodiments. Many details described in the following embodiments are for better understanding the present disclosure. However, those skilled in the art can realize with minimal effort that some of these features can be omitted in different cases or be replaced by other elements, materials and methods. For clarity some operations related to the present disclosure are not shown or illustrated herein so as to prevent the core from being overwhelmed by excessive descriptions. For those skilled in the art, such operations are not necessary to be explained in detail, and they can fully understand the related operations according to the description in the specification and the general technical knowledge in the art.

In addition, the features, operations or characteristics described in the specification may be combined in any suitable manner to form various embodiments. At the same time, the steps or actions in the described method can also be sequentially changed or adjusted in a manner that can be apparent to those skilled in the art. Therefore, the various sequences in the specification and the drawings are only for the purpose of describing a particular embodiment, and are not intended to be an order of necessity, unless otherwise stated one of the sequences must be followed.

The serial numbers of components herein, such as “first”, “second”, etc., are only used to distinguish the described objects and do not have any order or technical meaning. The terms “connected”, “coupled” and the like here include direct and indirect connections (coupling) unless otherwise specified.

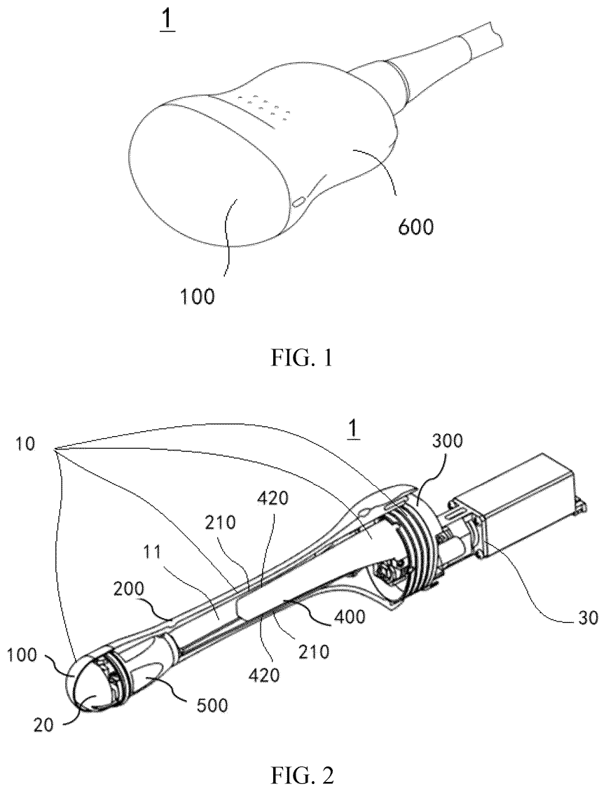

To reduce the weight of existing ultrasonic probes, specifically addressing the issue of excessive weight due to the large volume of coupling fluid in 3D or 4D ultrasonic probes, examples of an ultrasonic probe 1 are provided in some embodiments of the present disclosure, as shown in FIGS. 1 and 2.

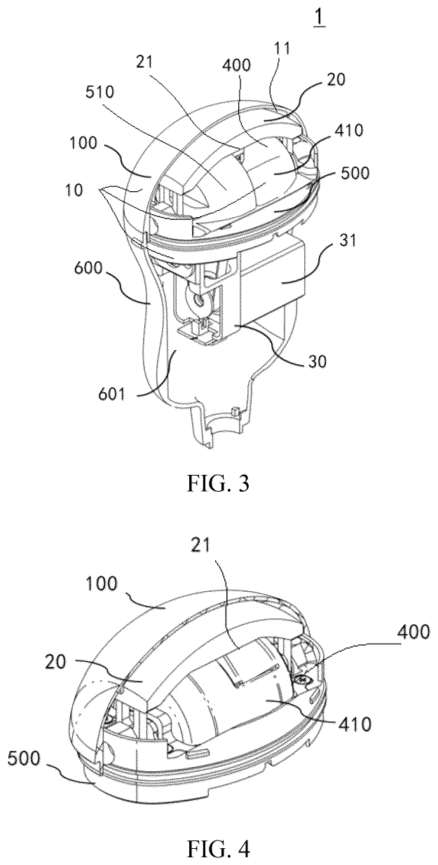

As shown in FIGS. 2 and 3, in some embodiments, the ultrasonic probe 1 comprises a coupling fluid cavity structure 10 that encloses a coupling fluid cavity 11, a transducer 20 and a transducer driving component 30. The transducer 20 is configured to transmit and receive ultrasonic signals, and is disposed in a swingable manner within the coupling fluid cavity 11. The transducer driving component 30 is configured to drive the transducer 20 to swing within a certain angle range. The transducer 20 scans and images a human body at each angle within its swing range, obtaining two-dimensional ultrasonic images (B-mode images), which may be subsequently utilized to construct three-dimensional (3D) or four-dimensional (4D) images of human tissues.

The coupling fluid cavity structure 10 is configured to enclose the coupling fluid cavity 11 that is filled inside with a coupling fluid. In different types of ultrasonic probes 1, the coupling fluid cavity structure 10 may be enclosed by different components. For example, as shown in FIG. 2, in some embodiments, the coupling fluid cavity structure 10 includes an acoustic window 100, a connecting housing 200, a probe base 300, and a coupling fluid compensating member 400, which together enclose the coupling fluid cavity 11. For another example, as shown in FIGS. 3 and 4, in some embodiments, the coupling fluid cavity structure 10 includes an acoustic window 100, a coupling fluid compensating member 400, and a transducer base 500, which together enclose the coupling fluid cavity 11. Of course, in other embodiments, the coupling fluid compensating member 400 may be excluded based on the aforementioned embodiments. Furthermore, various alternative configurations of the coupling fluid cavity structure 10 may be provided, which can be referred to in the prior art.

In order to reduce the size of the coupling fluid cavity 11 without affecting the normal swing of the transducer 20, and subsequently reduce the volume of the coupling fluid filled within the coupling fluid cavity 11, in some embodiments, a portion of the coupling fluid cavity structure 10 is configured as a profiling structure that protrudes towards the interior of the coupling fluid cavity 11. The profiling structure may be at least one of the coupling fluid compensating member 400, the transducer base 500, the acoustic window 100, the connecting housing 200, the probe base 300 and other components, all of which are components that constitute the coupling fluid cavity structure 10. For example, it may be, but not limit to, the coupling fluid compensating member 400 shown in FIGS. 2 and 3, and/or the protruding part 510 of the transducer base 500 shown in FIG. 3.

The profiling structure itself forms part of the coupling fluid cavity 11 and protrudes towards the interior of the coupling fluid cavity 11, occupying a portion of the original space within the coupling fluid cavity 11, thereby reducing the volume of the newly formed coupling fluid cavity 11 and consequently reducing the volume of the coupling fluid required to fill it. In order to maximize the volume of the profiling structure without affecting the normal swing of the transducer 20 and ensuring the presence of coupling fluid between the transducer 20 and the acoustic window 100, the shape of the profiling structure is designed to compress the size of the coupling fluid cavity 11 as much as possible. In some embodiments, the shape of the profiling structure is profiled against the swing trajectory of the transducer 20 and/or the surfaces, other than the profiling structure, of the walls of the coupling fluid cavity 11. The profiling structure is configured to closely conform to the swing trajectory of the transducer 20 or other structures, reducing redundant space within the coupling fluid cavity 11, and further decreasing the volume of the coupling fluid cavity 11.

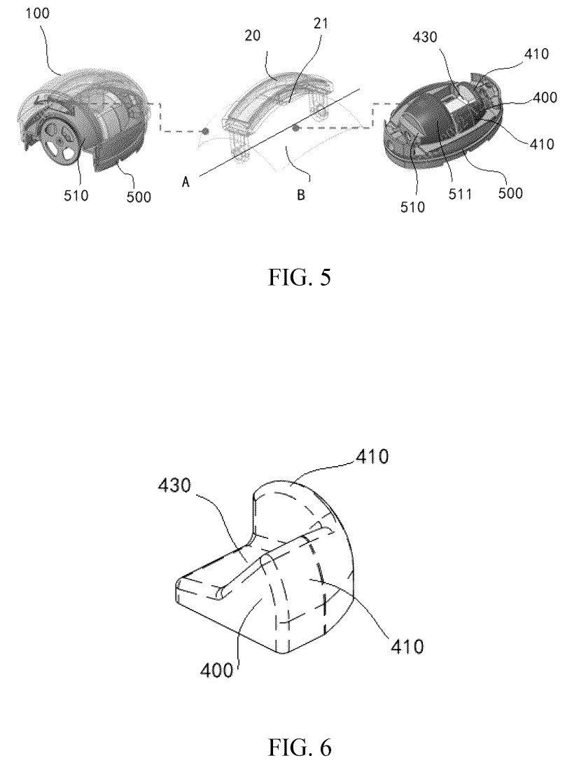

Specifically, in some embodiments, the transducer 20 generates a swing trajectory during its swing. This swing trajectory is a virtual shape, specifically representing the space traversed by the transducer 20 during its swing, or in other words, the space that needs to be reserved for the swing of the transducer 20. Depending on the shape of the transducer 20, the resulting swing trajectory varies, which may be a standard shape or an irregular shape. For example, in the embodiment illustrated in FIG. 5, the transducer 20 is in the shape of an arc structure, when rotated along its swing axis A, the swing trajectory is a trajectory region formed by the arcuate structure rotating around the swing axis A. At least a portion of the surface of the swing trajectory that faces the profiling structure (as indicated by B in FIG. 5) serves as a profiled surface (which may include, but is not limited to, the first profiled surface, the second profiled surface and/or the third profiled surface mentioned later). The profiling structure has at least a profiling surface (which may include, but is not limited to, the first profiling surface, the second profiling surface and the third profiling surface) that faces the profiled surface. The profiling surface and the profiled surface are of the same shape and disposed opposite to each other. That is, in the embodiment, the profiling surface of the profiling structure is made to fit as closely as possible with the swing trajectory of the transducer 20, so as to maximize the compression of the coupling fluid cavity 11 by the profiling structure without affecting the swing of the transducer 20, thereby making the coupling fluid cavity 11 smaller.

Additionally or alternatively, at least a portion of the surfaces, excluding the profiling structure, of the walls of the coupling fluid cavity 11 serves as the profiled surface. The profiling surface and the profiled surface are of the same shape and disposed opposite to each other. The surfaces, excluding the profiling structure, of the walls of the coupling fluid cavity 11 may be at least one of the coupling fluid compensating member 400, the transducer base 500, the acoustic window 100, the connecting housing 200, the probe base 300 and other components, all of which are components that constitute the coupling fluid cavity structure 10. That is, in this embodiments, the profiling surface of the profiling structure is made to fit as closely as possible with the surfaces, excluding the profiling structure, on the walls of the coupling fluid cavity 11, so as to maximize the compression of the coupling fluid cavity 11 by the profiling structure without affecting the swing of the transducer 20, thereby making the coupling fluid cavity 11 smaller. For example, in the embodiment shown in FIG. 2, the profiling structure conforms to the inner wall of the connecting housing 200.

In this embodiments, the profiling surface is configured with reference to the shape of the profiled surface, achieving an effect similar to that of obtaining the profiling surface by offsetting the profiled surface by a certain distance. This ensures that the profiled surface and the profiling surface have the same shape, such as the same size and variation trend (e.g., both being planar, folded, curved or a combination thereof), allowing the profiled surface and the profiling surface to be positioned as close as possible to each other.

In some embodiments, the profiling surface corresponds to the profiled surface, with the profiling surface being one or more, and the corresponding profiled surface also being one or more.

In some embodiments, the shortest distance from any point on the profiling face to its corresponding point on the conformed surface is equal, ensuring that the shape of the profiled surface and the shape of the corresponding profiling surface are identical.

According to the ultrasonic probe 1 provided in these embodiments, since a portion of its coupling fluid cavity structure 10 is a profiling structure that protrudes towards the interior of the coupling fluid cavity 11, and at least a portion of the surface of the swing trajectory of the transducer 20 that faces the profiling structure serves as a profiled surface, and/or at least a portion of the surfaces, excluding the profiling structure, of the cavity walls of the coupling fluid cavity 11, serves as a profiled surface, as well as the shape of the profiling surface being designed based on the shape of the profiled surface to ensure that the two surfaces are of the same shape, the profiling structure can thus be as close as possible to the swing trajectory of the transducer 20 or to the surfaces, excluding the profiling structure, of the cavity wall of the coupling fluid cavity 11, without affecting the swing of the transducer 20. This can reduce the size of the coupling fluid cavity 11, and consequently the volume of the coupling fluid, thereby lightening the weight of the ultrasonic probe 1.

In some embodiments, to better exploit the characteristic of identical shapes between the profiling surface and the profiled surface, the shortest distance between any point on the first profiling surface and the corresponding point on the first profiled surface is less than or equal to 10 mm. Obviously, a smaller distance indicates that the profiling surface is closer to the profiled surface, thereby causing increased compression of the coupling fluid cavity 11 by the profiling structure, leading to a smaller coupling fluid cavity 11.

Furthermore, in some embodiments, the shortest distance between any point on the profiling surface and the corresponding point on the profiled surface is less than or equal to 2 mm. Alternatively, the shortest distance between any point on the profiling surface and the corresponding point on the profiled surface is less than or equal to 1 mm, allowing the profiling surface to be even closer to the corresponding profiled surface.

Furthermore, considering the need for the profiling structure to provide clearance for other components or structures within the coupling fluid cavity 11, in some embodiments, the profiling surface may be an entire face in one or more directions on the profiling structure, or it may be a local region on one or more side surfaces. In other words, the profiling structure includes a clearance space 430, which may serve to provide clearance for other components or structures within the coupling fluid cavity 11. At this point, the profiled surface may correspond to the profiling surface, and may also be the entire surface in one or more directions or a local region on one or more side surfaces.

The following provides more specific embodiments to illustrate the inventive concept described above.

As shown in FIGS. 2-4, in some embodiments, the profiling structure comprises at least a coupling fluid compensating member 400 that is capable of adaptive deformation in response to the pressure exerted by the coupling fluid on the coupling fluid compensating member 400. For example, in some embodiments, the coupling fluid compensating member 400 may be made of an elastic, soft material, particular one that does not react with the coupling fluid or allow oil leakage, such as neoprene rubber. With the same volume, the weight of the coupling fluid compensating member 400 is less than that of the coupling fluid, and when the coupling fluid experiences volume changes due to temperature variations, the coupling fluid compensating member 400 can adaptively adjust, automatically expanding tor contracting the size of the coupling fluid cavity 11. The coupling fluid compensating member 400 may be either a solid structure or a hollow structure. Typically, the internal cavity of the hollow structure is empty, thereby making it more beneficial for reducing the overall weight of the ultrasonic probe 1.

As shown in FIGS. 3 and 4, in some specific embodiments, the ultrasonic probe 1 includes the transducer 20, the acoustic window 100, the transducer base 500, the coupling fluid compensating member 400 (the number of which is at least one), and the transducer driving component 30.

The transducer 20 is mounted on the transducer base 500. The acoustic window 100, the transducer base 500, and the coupling fluid compensating member 400 enclose the coupling fluid cavity 11. It is noted that, in this embodiment, the structure enclosing the coupling fluid cavity 11 is not limited to the acoustic window 100, the transducer base 500, and the coupling fluid compensating member 400; other components may be included based on design requirements or additional functional needs. The coupling fluid cavity 11 is filled with coupling fluid; and the transducer 20 is disposed in a swingable manner within the coupling fluid cavity 11.

As shown in FIGS. 3-7, the transducer 20 generates the swing trajectory during its swing; at least a portion of the coupling fluid compensating member 400 is disposed between the swing trajectory and the transducer base 500; at least a portion of the surface of the swing trajectory that faces the coupling fluid compensating member 400 (which may be one or several surfaces) serves as the first profiled surface (not labeled in the figures). The coupling fluid compensating member 400 comprises at least one first profiling surface 410 that faces the first profiled surface. The first profiling surface 410 and the first profiled surface are identical in shape and are disposed opposite to each other.

According to the ultrasonic probe 1 provided in the embodiment shown in FIG. 3, since at least a portion of the surface of the swing trajectory of the transducer 20 that faces the coupling fluid compensating member 400 is configured as a first profiled surface, and the coupling fluid compensating member 400 has at least a first profiling surface 410 that faces the first profiled surface, as well as the shape of the first profiling surface 410 being designed based on the shape of the first profiled surface to ensure that the two surfaces are of the same shape, the coupling fluid compensating member 400 can thus be as close as possible to the swing trajectory of the transducer 20 without affecting the swing of the transducer 20. This can reduce the size of the coupling fluid cavity 11, and consequently the volume of the coupling fluid, thereby lightening the weight of the ultrasonic probe 1.

In some embodiments, the shortest distance between any point on the first profiling surface 410 and the corresponding point on the first profiled surface is equal.

In some embodiments, the shortest distance between any point on the first profiling surface 410 and the corresponding point on the first profiled surface is equal.

In some embodiments, the shortest distance between any point on the first profiling surface 410 and the corresponding point on the first profiled surface is less than or equal to 10 mm. Alternatively, in some embodiments, the shortest distance between any point on the first profiling surface 410 and the corresponding point on the first profiled surface is less than or equal to 2 mm. Alternatively, in some embodiments, the shortest distance between any point on the first profiling surface 410 and the corresponding point on the first profiled surface is less than or equal to 1 mm, allowing the first profiling surface 410 to be even closer to the first profiled surface.

Furthermore, as shown in FIGS. 3 and 4, in some embodiments, the surface of the transducer 20 facing the transducer base 500 is defined as a transducer bottom surface; at least a portion of the transducer bottom surface is concave towards the interior of the transducer 20 to form an inner concave surface 21; and at least a portion of the coupling fluid compensating member 400 extends into a region enclosed by the inner concave surface 21. During the swing of the transducer 20, the swing trajectory generated by the inner concave surface 21 is concave inward as a whole, allowing the coupling fluid compensating member 400 to be accommodated within this concave region, thereby reducing the overall volume of the ultrasonic probe 1. The surface of the swing trajectory generated by the inner concave surface 21 that faces the coupling fluid compensating member 400 may have one or more first profiled surfaces. At least a portion of the first profiling surface 410 of the coupling fluid compensating member 400 can conform to these first profiled surfaces.

As shown in FIGS. 5 and 6, in some embodiments, the first profiled surface, which is the surface of the swing trajectory of the transducer 20 that faces the coupling fluid compensating member 400, is an arcuate surface having the swing axis A of the transducer 20 as its central axis; and in this regard, the first profiling surface 410 is the arcuate surface having the swing axis A of the transducer 20 as its central axis. Of course, the first profiled surface and the first profiling surface 410 are not limited to this arcuate structure; they may be other structures, even irregular shapes.

As shown in FIG. 6, in some embodiments, the first profiling surface 410 on the coupling fluid compensating member 400 is disposed on its side, and the clearance space 430 is left beside the first profiling surface 410 to accommodate other components.

As shown in FIGS. 3 and 7-9, in some embodiments, the ultrasonic probe 1 further includes a handle housing 600. The transducer base 500 and the handle housing 600 enclose a drive component installation cavity 601; and the transducer driving component 30 is disposed within the drive component installation cavity 601. Apart from the necessary transmission connection between the transducer driving component 30 and the transducer 20, the transducer base 500 separates the drive component installation cavity 601 from the coupling fluid cavity 11 to prevent the coupling fluid flowing into the drive component installation cavity 601. The transducer base 500 has a vent 520 (see FIG. 8) communicating with the drive component installation cavity 601; and the coupling fluid compensating member 400 has a cavity structure. The cavity structure is sealably covered over the vent 520 of the transducer base 500 and communicates with the vent 520, thereby communicating the inner cavity of the cavity structure with the drive component installation cavity 601. The drive component installation cavity 601 can be connected to or isolated from the atmospheric environment. Gas within the drive component installation cavity 601 or from the atmospheric environment can enter the cavity structure of the coupling fluid compensating member 400 through the vent 520, providing pressure support to the coupling fluid compensating member 400. Moreover, since the coupling fluid compensating member 400 is communicated with the drive component installation cavity 601, when the density and volume of the coupling fluid in the coupling fluid cavity 11 change, the coupling fluid compensating member 400 can adaptively change its volume under the action of the gas within its cavity structure, thereby compensating for the coupling fluid. Of course, in other embodiments, the coupling fluid compensating member 400 can also be a sealed cavity structure filled with gas or liquid. Alternatively, the coupling fluid compensating member 400 may be a solid structure made of elastic, soft material.

Furthermore, as shown in FIGS. 3-5 and 7-9, in some embodiments, the transducer base 500 includes a protruding part 510 that protrudes towards the transducer 20. This protruding part 510 can also serve as a type of profiling structure. The protruding part 510 is either integrally formed with the transducer base 500 or fixedly connected to it. The protruding part 510 can be made of a rigid material to ensure structural stability.

The protruding part 510 includes a second profiling surface 511, and at least a portion of the surface(s) of the swing trajectory that faces the protruding part 510 (which may be one or several surfaces) serves as a second profiled surface (not labeled in the figures). The second profiling surface 511 is identical in shape to the second profiled surface and is positioned opposite to it. In this embodiment, the shape of the second profiling surface 511 is designed based on the shape of the second profiled surface to ensure their identical shapes. This allows the protruding part 510 to be positioned as close as possible to the swing trajectory of the transducer head 20 without affecting its swing, thereby reducing the size of the coupling fluid cavity 11, decreasing the volume of the coupling fluid, and ultimately lightening the weight of the ultrasonic probe 1.

In some embodiments, the shortest distance between any point on the second profiling surface 511 and the corresponding point on the second profiled surface is equal.

In some embodiments, the shortest distance between any point on the second profiling surface 511 and the corresponding point on the second profiled surface is less than or equal to 10 mm. Alternatively, in some embodiments, the shortest distance between any point on the second profiling surface 511 and the corresponding point on the second profiled surface is less than or equal to 2 mm. Alternatively, in some embodiments, the shortest distance between any point on the second profiling surface 511 and the corresponding point on the second profiled surface is less than or equal to 1 mm, allowing the second profiling surface 511 to be even closer to the second profiled surface.

Furthermore, in some embodiments, similar to the first profiling surface 410 mentioned above, during the swing of the transducer 20, the swing trajectory generated by the inner concave surface 21 is concave inward as a whole, allowing the protruding part 510 to be accommodated within this concave region. The surface of the swing trajectory generated by the inner concave surface 21 that faces the protruding part 510 may have one or more second profiled surfaces. At least a portion of the second profiling surface 511 of the protruding part 510 can conform to these second profiled surfaces.

As shown in FIGS. 3, 5 and 7, in some embodiments, the second profiled surface, which is the surface of the swing trajectory of the transducer 20 that faces the coupling fluid compensating member 400, is an arcuate surface having the swing axis A of the transducer 20 as its central axis; and in this regard, the second profiling surface 511 is the arcuate surface having the swing axis A of the transducer 20 as its central axis. Of course, the second profiled surface and the second profiling surface 511 are not limited to this arcuate structure; they may be other structures, even irregular shapes.

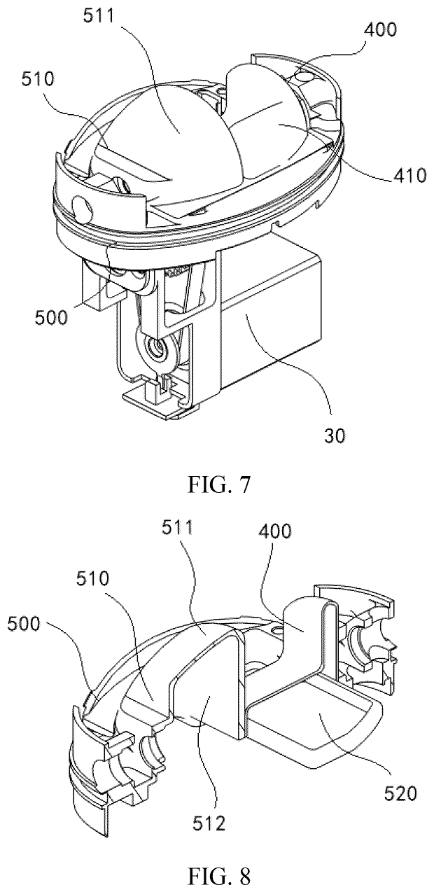

Furthermore, when the transducer base 500 has a protruding part 510, in addition to enabling profiling design, the transducer driving component 30 can be integrated with the protruding part 510 to provide a more compact structure design conducive to reducing the volume of the ultrasonic probe 1. In some embodiments, as shown in FIGS. 7-9, the transducer driving component 30 is disposed on the side of the transducer base 500 away from the transducer 20; a side of the protruding part 510 that faces the transducer driving component 30 forms a accommodation cavity 512; and at least a portion of the transducer driving component 30 is accommodated within the accommodation cavity 512. Accordingly, a portion of the transducer driving component 30 is accommodated within the protruding part 510, that is, a portion of the original space of the coupling fluid cavity 11 is utilized to accommodate the transducer driving component 30, thereby reducing the overall space occupied by both the coupling fluid cavity 11 and the transducer driving component 30, and consequently enabling the design of a smaller ultrasonic probe 1.

In some embodiments, the transducer driving component 30 comprises a drive motor and a transmission mechanism. The transmission mechanism is operatively connected to the drive motor, and the transducer 20 is operatively connected to the transmission mechanism. The motion generated by the drive motor is transmitted through the transmission mechanism to the transducer 20, thereby driving the transducer 20 to swing. A portion of the transmission mechanism is accommodated within the housing cavity 512 of the protruding part 510.

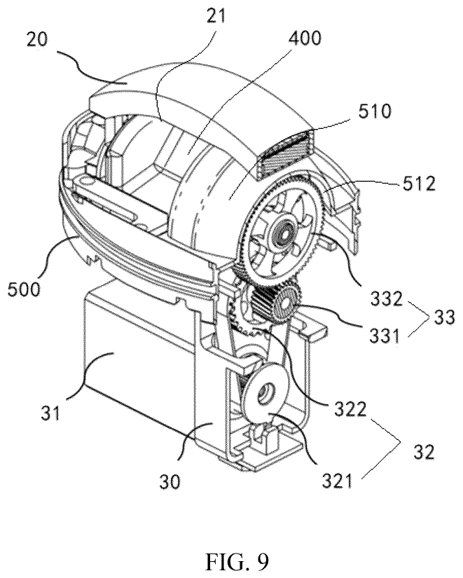

In some embodiments, as shown in FIG. 9, the transmission mechanism includes a synchronous belt pulley transmission mechanism 32 and a gear drive unit 33. The drive pulley 321 of the synchronous belt pulley transmission mechanism 32 is connected to the output end of the drive motor 31, and the driven pulley 322 of the synchronous belt pulley transmission mechanism 32 is connected to the input gear 331 of the gear drive unit 33. The output gear 332 of the gear drive unit 33 is operatively connected to the transducer 20. Specifically, the rotational axes of the motor output shaft, the drive pulley 321, the driven pulley 322, the input gear 331, the output gear 332, and the swing axis A of the transducer 20 are all parallel to each other. This arrangement allows the overall structure of the transmission mechanism to be roughly linear, facilitating the efficient accommodation of the output gear 332 within the housing cavity 512.

Of course, in other embodiments, the transducer driving component 30 may employ other existing structures to achieve the swing drive of the transducer 20.

Furthermore, to further enhance structural compactness, in some embodiments, as shown in FIG. 3, at least a portion of the surface of the transducer 20 that faces the transducer base 500 is concave towards the interior of the transducer 20, forming the inner concave surface 21; and the protruding part 510 extends into a region enclosed by the inner concave surface 21. This arrangement allows for a smaller overall structure of the transducer 20, the coupling fluid cavity 11 and the transducer driving component 30, thereby enabling the design of a smaller ultrasonic probe 1.

Of course, in some embodiments, as shown in FIG. 4, the transducer base 500 may exclude the protruding part 510, and the transducer driving component 30 is disposed below the transducer base 500, without protruding into the coupling fluid cavity 11. In this case, the coupling fluid compensating member 400 can be expanded to cover a large area of the transducer base 500, allowing for a larger design range for the first profiling surface 410.

Furthermore, in some other embodiments, the ultrasonic probe 1 may not include the coupling fluid compensating member 400. In this case, the ultrasonic probe 1 includes the transducer 20, the acoustic window 100, the transducer base 500 and the transducer driving component 30. The acoustic window 100 and the transducer base 500 enclose the coupling fluid cavity 11 that is filled inside with a coupling fluid; and the transducer 20 is disposed in a swingable manner within the coupling fluid cavity 11. Specifically, the transducer base 500 has a protruding part 510 that protrudes towards the transducer 20; the transducer 20 generates the swing trajectory during its swing motion; at least a portion of the surface of the swing trajectory that faces the protruding part 510 serves as the second profiled surface; and the protruding part 510 has the second profiling surface 511, where the second profiling surface 511 and the second profiled surface are identical in shape and disposed opposite to each other.

In the embodiments without the coupling fluid compensating member 400, the protruding part 510 can be designed as shown in the above embodiments. At least a portion of the surface of the swing trajectory of the transducer 20 that faces the protruding part 510 serves as the second profiled surface. The protruding part 510 has the second profiling surface 511. The shape of the second profiling surface 511 is designed based on the shape of the second profiled surface to ensure they are the same, allowing the protruding part 510 to be as close as possible to the swing trajectory of the transducer 20 without affecting its swing motion. This reduces the size of the coupling fluid cavity 11, decreases the volume of coupling fluid, and thereby lightens the weight of the ultrasonic probe 1.

In accordance with another aspect, in other types of the ultrasonic probes 1, as shown in FIG. 2, the ultrasonic probe 1 includes the probe base 300, the connecting housing 200, the acoustic window 100, the coupling fluid compensating member 400, the transducer base 500, the transducer 20 and the transducer driving component 30.

The connecting housing 200 is connected to the probe base 300, and the acoustic window 100 is connected to the connecting housing 200. The probe base 300, the acoustic window 100, the connecting housing 200, and the coupling fluid compensating member 400 enclose the coupling fluid cavity 11. Of course, in this embodiment, the structure enclosing the coupling fluid cavity 11 is not limited to the probe base 300, the acoustic window 100, the connecting housing 200, and the coupling fluid compensating member 400; other components may be added based on design requirements or additional functionalities. The coupling fluid cavity 11 is filled with coupling fluid; and the transducer base 500 is disposed within the coupling fluid cavity 11. Unlike previous embodiments, in this embodiment, the transducer base 500 is disposed within the coupling fluid cavity 11, meaning that the transducer base 500 is also surrounded by the coupling fluid.

At least a portion of the inner wall surface of the connecting housing 200 serves a third profiled surface (not labeled in the figure). The coupling fluid compensating member 400 has at least one third profiling surface 420 that faces the third profiled surface. The third profiling surface 420 and the third profiled surface are of the same shape and disposed opposite to each other.

In the ultrasonic probe 1 provided in this embodiment, the coupling fluid compensating member 400 does not conform to the swing trajectory of the transducer 20 but rather conforms to the inner wall surface 210 of the connecting housing 200. The shape of the third profiling surface 420 is designed based on the shape of the third profiled surface to ensure they are identical. This allows the coupling fluid compensating member 400 to be as close as possible to the inner wall surface 210 of the connecting housing 200 without affecting the swing motion of the transducer 20. This reduces the size of the coupling fluid cavity 11, decreases the volume of coupling fluid, and thereby lightens the weight of the ultrasonic probe 1.

The aforementioned first profiling surface 410, second profiling surface 511 and third profiling surface 420 are all profiling surfaces. For the convenience of description in the present disclosure, they are categorized as the first profiling surface 410, second profiling surface 511 and third profiling surface 420. There is no sequential order among the first, second, and third designations; that is, the presence of the third profiling surface 420 in some embodiments does not necessarily imply the presence of the first profiling surface 410 or the second profiling surface 511. Similarly, the same applies to the first profiling surface, second profiling surface and third profiling surface.

In some embodiments, the shortest distance between any point on the third profiling surface 420 and the corresponding point on the third profiled surface is equal.

In some embodiments, the shortest distance between any point on the third profiling surface 420 and the corresponding point on the third profiled surface is less than or equal to 10 mm. Alternatively, in some embodiments, the shortest distance between any point on the third profiling surface 420 and the corresponding point on the third profiled surface is less than or equal to 2 mm. Alternatively, in some embodiments, the shortest distance between any point on the third profiling surface 420 and the corresponding point on the third profiled surface is less than or equal to 1 mm, allowing the third profiling surface 420 to be even closer to the third profiled surface.

As shown in FIG. 2, in some embodiments, the connecting housing 200 is elongated to facilitate insertion into the body cavity of an object under examination. The coupling fluid compensating member 400 is mounted on the probe base 300 and extends into the connecting housing 200 along its length direction. The third profiling surface 420 is disposed on the side wall of the coupling fluid compensating member 400.

As shown in FIG. 2, in some embodiments, the ultrasonic probe 1 specifically can be an endocavity probe designed to be inserted into the body cavity of the object under examination. Of course, the ultrasonic probe 1 can also be other types of ultrasonic probes beyond the endocavity probe.

Based on the ultrasonic probe 1 shown in the above embodiments, the present disclosure further provides an ultrasonic device. The device includes an ultrasonic hos and the ultrasonic probe 1 as described in any of the aforementioned embodiments. The ultrasonic mainframe comprises a control unit that operates to control the operation of the ultrasonic probe 1. The ultrasonic device provided herein employs the ultrasonic probe 1 shown in the previous embodiments, enabling a reduction in the weight of the ultrasonic probe 1 without affecting the swing of the acoustic head 20.

The specific examples employed above to illustrate the present invention are for the purpose of facilitating understanding and are not intended to limit the invention. Those skilled in the art to which the present invention pertains, without departing from the basic principles of the present invention, may make various simple deductions, modifications or substitutions.

What is claimed is:

1. An ultrasonic probe, comprising:
a transducer configured to transmit and receive ultrasonic signals;
an acoustic window;
a transducer base, wherein the transducer is mounted on the transducer base;
a coupling fluid compensating member, wherein the acoustic window, the transducer base and the coupling fluid compensating member enclose a coupling fluid cavity for coupling fluid, and the transducer is disposed in a swingable manner within the coupling fluid cavity; and
a transducer driving component configured to drive the transducer to swing;
wherein a swing trajectory is generated while the transducer is driven to swing, at least a portion of the coupling fluid compensating member is disposed between the swing trajectory and the transducer base, at least a portion of a surface of the swing trajectory that faces the coupling fluid compensating member serves as a first profiled surface, and the coupling fluid compensating member has at least one first profiling surface that faces the first profiled surface, the first profiling surface and the first profiled surface are of a same shape and disposed opposite to each other.

2. The ultrasonic probe according to claim 1, wherein a shortest distance between any point on the first profiling surface and a corresponding point on the first profiled surface is equal.

3. The ultrasonic probe according to claim 2, wherein the shortest distance between any point on the first profiling surface and the corresponding point on the first profiled surface is less than or equal to 10 mm.

4. The ultrasonic probe according to claim 1, wherein a surface of the transducer facing the transducer base is defined as a transducer bottom surface, at least a portion of the transducer bottom surface is concave towards an interior of the transducer to form an inner concave surface, and at least a portion of the coupling fluid compensating member extends into a region enclosed by the inner concave surface.

5. The ultrasonic probe according to claim 1, wherein each of the at least one first profiling surface is an arc surface whose central axis is a swing axis of the transducer.

6. The ultrasonic probe according to claim 1, wherein the coupling fluid compensating member is made of an elastic, soft material, thereby allowing the coupling fluid compensating member to adaptively deform based on a pressure exerted by the coupling fluid on the coupling fluid compensating member.

7. The ultrasonic probe according to claim 6, further comprising a handle housing, wherein the transducer base and the handle housing enclose an installation cavity for the transducer driving component; the transducer base is provided with a vent connecting with the installation cavity for the transducer driving component, the coupling fluid compensating member has a cavity structure, and the cavity structure is covered over the vent of the transducer base and is connected with the vent, thereby connecting an inner cavity of the cavity structure with the installation cavity for the transducer driving component.

8. The ultrasonic probe according to claim 1, wherein the transducer base has a protruding part that protrudes towards the transducer, the protruding part has a second profiling surface, and at least a portion of a surface of the swing trajectory that faces the protruding part serves as a second profiled surface, the second profiling surface and the second profiled surface are of a same shape and disposed opposite to each other.

9. The ultrasonic probe according to claim 8, wherein the transducer driving component is disposed on a side of the transducer base away from the transducer, a side of the protruding part that faces the transducer driving component forms an accommodation cavity, and at least a portion of the transducer driving component is accommodated within the accommodation cavity.

10. The ultrasonic probe according to claim 9, wherein at least a portion of a surface of the transducer that faces the transducer base is concave inward and forms an inner concave surface within the transducer, and the protruding part extends into a region enclosed by the inner concave surface.

11. An ultrasonic probe, comprising:
a transducer configured to transmit and receive ultrasonic signals;
an acoustic window;
a transducer base, wherein the transducer is mounted on the transducer base, the acoustic window and the transducer base enclose a coupling fluid cavity for coupling fluid, and the transducer is disposed in a swingable manner within the coupling fluid cavity; and
a transducer driving component configured to drive the transducer to swing;
wherein the transducer base has a protruding part that protrudes towards the transducer, a swing trajectory is generated while the transducer is driven to swing, at least a portion of a surface of the swing trajectory that faces the protruding part serves as a second profiled surface, and the protruding part has a second profiling surface, the second profiling surface and the second profiled surface are of a same shape and disposed opposite to each other.

12. The ultrasonic probe according to claim 11, wherein a shortest distance between any point on the second profiling surface and a corresponding point on the second profiled surface is equal.

13. The ultrasonic probe according to claim 11, wherein the transducer driving component is disposed on a side of the transducer base away from the transducer, at least a portion of a surface of the transducer that faces the transducer base is concave inward and forms an inner concave surface within the transducer, the protruding part extends into a region enclosed by the inner concave surface; a side of the protruding part that faces the transducer driving component forms an accommodation cavity, and at least a portion of the transducer driving component is accommodated within the accommodation cavity.

14. An ultrasonic probe, comprising:
a probe base;
a connecting housing that is connected to the probe base;
an acoustic window that is connected to the connecting housing;
a coupling fluid compensating member, wherein the probe base, the acoustic window, the connecting housing and the coupling fluid compensating member enclose a coupling fluid cavity for coupling fluid;
a transducer base that is disposed within the coupling fluid cavity;
a transducer configured to transmit and receive ultrasonic signals, wherein the transducer is mounted on the transducer base and is capable of swinging within the coupling fluid cavity; and
a transducer driving component configured to drive the transducer to swing;
wherein at least a portion of an inner side wall of the connecting housing serves as a third profiled surface, the coupling fluid compensating member has at least one third profiling surface that faces the third profiled surface, the third profiling surface and the third profiled surface are of a same shape and disposed opposite to each other.

15. The ultrasonic probe according to claim 14, wherein a shortest distance between any point on the third profiling surface and a corresponding point on the third profiled surface is equal.

16. The ultrasonic probe according to claim 15, wherein the shortest distance between any point on the third profiling surface and the corresponding point on the third profiled surface is less than or equal to 10 mm.

17. The ultrasonic probe according to claim 15, wherein the shortest distance between any point on the third profiling surface and the corresponding point on the third profiled surface is less than or equal to 2 mm.

18. The ultrasonic probe according to claim 14, wherein the coupling fluid compensating member is made of an elastic, soft material, thereby allowing the coupling fluid compensating member to adaptively deform based on a pressure exerted by the coupling fluid on the coupling fluid compensating member.

19. The ultrasonic probe according to claim 14, wherein the connecting housing is elongated, and the coupling fluid compensating member is mounted on the probe base and extends into the connecting housing along a length direction of the connecting housing.

20. The ultrasonic probe according to claim 14, wherein the ultrasonic probe is an endocavity probe.
